(19) United States
(12) Patent Application Publication
(10) Pub. No.: US 2024/0398421 A1
(43) Pub. Date: Dec. 5, 2024

(54) REMOVABLE OVER-THE-SCOPE CLIP

(71) Applicant: BOSTON SCIENTIFIC SCIMED, INC., Maple Grove, MN (US)

(72) Inventors: Jennifer LEWITZKY, Lexington, MA (US); Colby HARRIS, Norfolk, MA (US); Austin Grant JOHNSON, Worcester, MA (US); Allison KUMARASENA, Wellesley, MA (US)

(21) Appl. No.: 18/642,449

(22) Filed: Apr. 22, 2024

Related U.S. Application Data
(60) Provisional application No. 63/505,193, filed on May 31, 2023.

(51) Int. Cl. A61B 17/122; A61B 17/00; A61B 17/128

(52) U.S. Cl. CPC A61B 17/122; A61B 17/1285; A61B 2017/00584; A61B 2017/00623

(57) ABSTRACT

A clip includes first and second jaws, first and second hinges, and a connection portion. Each of the jaws extends along a curve from a first end to a second end. The first hinge connects the first ends and the second hinge connects the second ends so that the jaws are movable between at open configuration, in which the jaws are separated from one another, and a closed configuration, in which the jaws are drawn toward one another to grip tissue therebetween. The first and/or second hinges is/are biased to draw the clip toward the closed configuration. The portion connects an end of each of the hinges to a corresponding one of the first and second ends. The connecting includes an engaging feature engaging via a through-the scope device to extract the clip from a tissue over which it has been clipped upon a deployment thereof.

### PRIORITY CLAIM

The present disclosure claims priority to U.S. Provisional Patent Application Ser. No. 63/505,193 filed May 31, 2023; the disclosure of which is incorporated herewith by reference.

### FIELD

The present disclosure relates to endoscopic devices and, in particular, relates to endoscopic clipping devices for treating tissue along the gastrointestinal tract.

### BACKGROUND

Physicians have become more willing to perform aggressive interventional and therapeutic endoscopic gastrointestinal (GI) procedures, which may increase the risk of perforating the wall of the GI tract or may require closure of the GI tract wall as part of these procedures. Such procedures may include, for example, the removal of large lesions, tunneling under the mucosal layer of the GI tract to treat issues below the mucosa, full thickness removal of tissue, treatment of issues of other organs by passing out of the GI tract a device inserted into the body through the GI tract, and endoscopic treatment/repair of post-surgical issues (e.g., post-surgical leaks, breakdown of surgical staple lines, and anastomotic leaks). Currently, tissue may be treated via endoscopic closure devices such as through-the scope clips or over-the-scope clips. Over-the-scope clips may be particularly useful for achieving closure of larger tissue defects. These endoscopic closure devices can save costs for the hospital and may provide benefits for the patient.

In some cases, however, it may be imperative for a physician to remove a previously deployed over-the-scope clip. An improperly placed clip may, for example, cause bleeding and perforations, or obstruct/traumatize neighboring tissue or organs. Although methods for removing deployed over-the-scope clips currently exist, these generally involve cutting the clip in half utilizing a conducting wire, an endoscopic mucosal resection (EMR) device, or an endoscopic submucosal dissection (ESD) device. In addition, in some cases, if the clip extends away from tissue so that it is reachable, grasping forceps may be used to pull the clip off of the tissue.

Cutting and extracting the clip, however, is tedious and time consuming. After placement and/or deployment of a clip, removal of the clip generally requires that the endoscope be removed from the body and fitted with accessories configured for the removal of the clip—e.g., a retrieval cap, conductive cutter, etc. When a clip is to be severed (e.g., using a conductive cutter) to free it from clipped tissue, a user must, after severing the clip, remove the conductive cutter from a working channel of the endoscope and then insert a grasper through the working channel to grasp the cut clip and draw the cut portions of the clip into a retrieval cap for removal from the body. It is noted that the severed pieces of the clip must generally be extracted from the body one at a time so that the grasper and a first part of the clip must be removed from the body and then the grasper must be reinserted through the working channel to remove the second part of the severed clip. This removal system along with EMRs and ESDs almost always send voltages through the clip into the clipped tissue potentially damaging the target tissue (tissue a user wants to clip) and/or healthy tissue surrounding the target tissue. While grasping forces do not carry any current, the process of pulling or pulling the clip off of clipped tissue may cause serious bleeds/perforations, etc., as would be understood by those skilled in the art.

### SUMMARY

The present disclosure relates to an over-the-scope clip which includes a first jaw and a second jaw, each of the first and second jaws extending along a curve from a first end to a second end. The clip also includes a first hinge connecting the first ends of the first and second jaws and a second hinge connecting the second ends of the first and second jaws so that the first and second jaws are movable between at open configuration, in which the first and second jaws are separated from one another, and a closed configuration, in which the first and second jaws are drawn toward one another to grip tissue therebetween. At least one of the first and second hinges is biased to draw the clip toward the closed configuration.

In addition, the clip includes a connecting portion connecting an end of each of the first and second hinges to a corresponding one of the first and second ends of each of the first and second jaws. The connecting portion includes an engaging feature configured to be engaged via a through-the scope device to extract the clip from a tissue over which it has been clipped upon a deployment thereof.

In an embodiment, the engaging feature includes a first notch extending along a portion of an exterior edge of the connecting portion in alignment with a corresponding one of the first and second jaws.

In an embodiment, the engaging feature includes a second notch extending along a portion of the exterior edge of the connecting portion in alignment with a corresponding one of the first and second hinges.

In an embodiment, the first and second notches are configured as slots which are aligned with one another.

In an embodiment, the engaging feature is a protrusion extending from an exterior surface of the connecting portion, the protrusion including an enlarged head connected to the exterior surface via a stem.

In an embodiment, the stem extends substantially perpendicular to the exterior surface.

In an embodiment, the stem includes an L-shaped bend.

In an embodiment, the engaging feature includes an opening extending through the corresponding one of the connecting portions.

In an embodiment, each of the first and second hinges includes a curve extending toward and between the first and second jaws.

In an embodiment, each of the first and second jaws include a plurality of teeth configured to grip tissue received therebetween.

In addition, the present disclosure relates to an over-the-scope clip which includes a first jaw and a second jaw. A first end of the first jaw is connected to a first end of the second jaw via a first hinge and a second end of the first jaw is connected to a second end of the second jaw via a second hinge so that the first and second jaws are movable between an open configuration, in which the first and second jaws are separated from one another, and a closed configuration, in which the first and second jaws are drawn toward one another to grip tissue therebetween. At least one of the first and second hinges is biased to draw the clip toward the closed configuration. The clip also includes a first engaging feature extending from an edge of the first jaw which faces away from the second jaw and a second engaging feature extending from an edge of the second jaw which faces away from the first jaw.

In an embodiment, each of the first and second engaging features include a protrusion including an enlarged head connecting to the edge of a corresponding one of the first and second jaws via a stem.

In an embodiment, each of the first and second jaws extend along a curve corresponding to a surface of an end cap of an endoscope over which the clip is to be mounted.

In an embodiment, each of the first and second hinges includes a curve extending toward and between the first and second jaws.

In an embodiment, each of the first and second jaws include a plurality of teeth configured to grip tissue received therebetween.

In addition, the present disclosure relates a method for extracting a deployed over-the-scope clip from a body. The method includes inserting a snare device through a working channel of an endoscope via which the clip was clipped over a tissue and deployed; looping a wire of the snare device over two or more engaging features of first and second jaws of the clip so that the wire engages the one or more engaging features, the clip including a first hinge connecting first ends of the first and second jaws and a second hinge connecting second ends of the first and second jaws, ends of the first and second hinges connected to a corresponding one of the first and second ends of the first and second jaws via connecting portions including the engaging features; tightening the wire to draw the two or more engaging features toward one another so that one of the first and second hinges is compressed to move the clip from a closed configuration, in which the first and second jaws are moved toward one another to clip a tissue therebetween, toward an open configuration, in which the first and second jaws are moved away from one another to release the tissue gripped therebetween; drawing the clip proximally away from the released tissue and against a distal end of the endoscope; and removing the clip from the body, simultaneously with the endoscope.

In an embodiment, each of the engaging features includes a first notch extending along a portion of an exterior edge of the connecting portion in alignment with a corresponding one of the first and second jaws and a second notch extending along a portion of the exterior edge of the connecting portion in alignment with a corresponding one of the first and second hinges.

In an embodiment, the wire is looped over four connecting portions.

In an embodiment, the wire is looped over the engaging features of two diagonally positioned connecting portions, a first one of the connecting portions at the first end of the first jaw and a second one of the connecting portions at the second end of the second jaw.

In an embodiment, the wire is looped over the engaging features of two opposing connecting portions, a first one of the opposing connecting portions at the first end of the first jaw and a second one of the opposing connecting portions at the first end of the second jaw.

### BRIEF DESCRIPTION

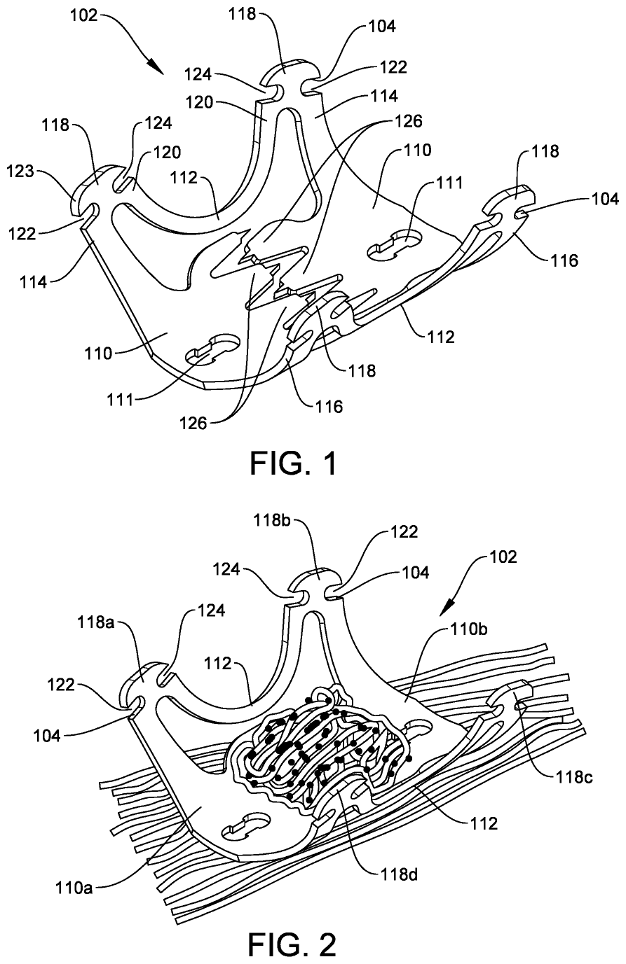

FIG. 1 shows a perspective view of a clip according to an exemplary embodiment of the present disclosure;

FIG. 2 shows a perspective view of the clip according to FIG. 1, the clip clipped over tissue;

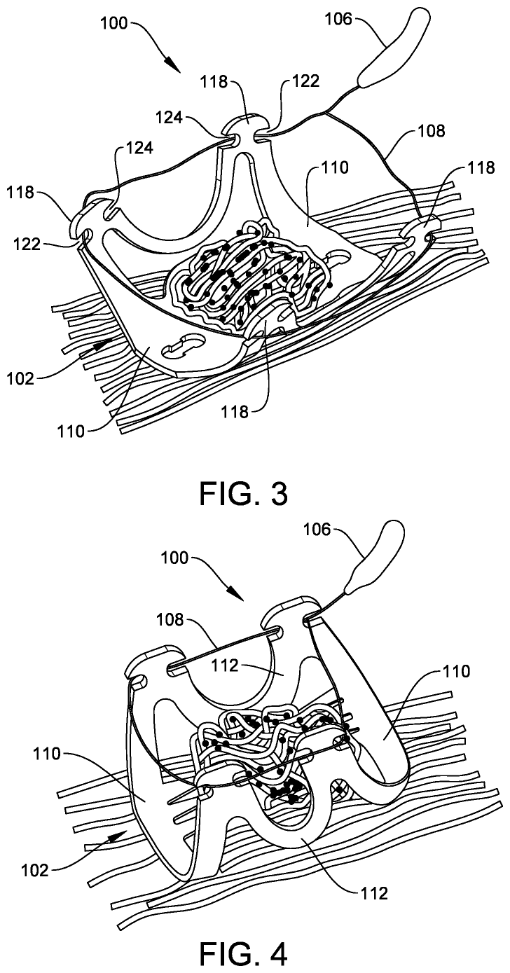

FIG. 3 shows a perspective view of the clip of FIG. 1, the clip engaged via a loop of a snare device according to an exemplary method of the present disclosure;

FIG. 4 shows a perspective view of the clip according to FIG. 1, the clip moved from a closed configuration toward an open configuration via a tightening of the loop of the snare device to release the tissue gripped thereby;

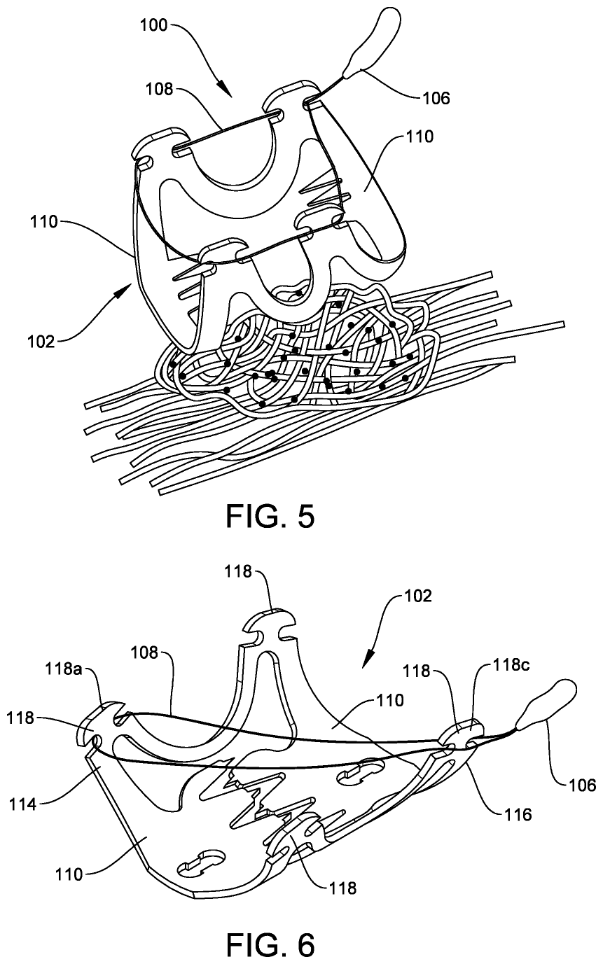

FIG. 5 shows a perspective view of the clip according to FIG. 1, the clip moved away from the released tissue;

FIG. 6 shows a perspective view of the clip according to FIG. 1, engaging features of the clip looped via a snare device according to another exemplary method;

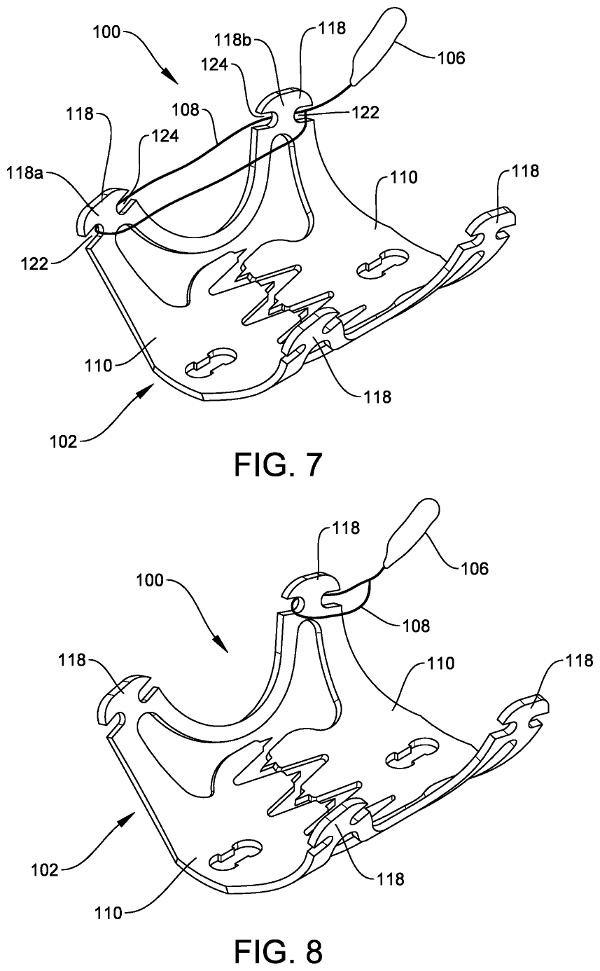

FIG. 7 shows a perspective view of the clip according to FIG. 1, engaging features of the clip looped via a snare device according to another exemplary method;

FIG. 8 shows a perspective view of the clip according to FIG. 1, an engaging feature of the clip looped via a snare device according to yet another exemplary method;

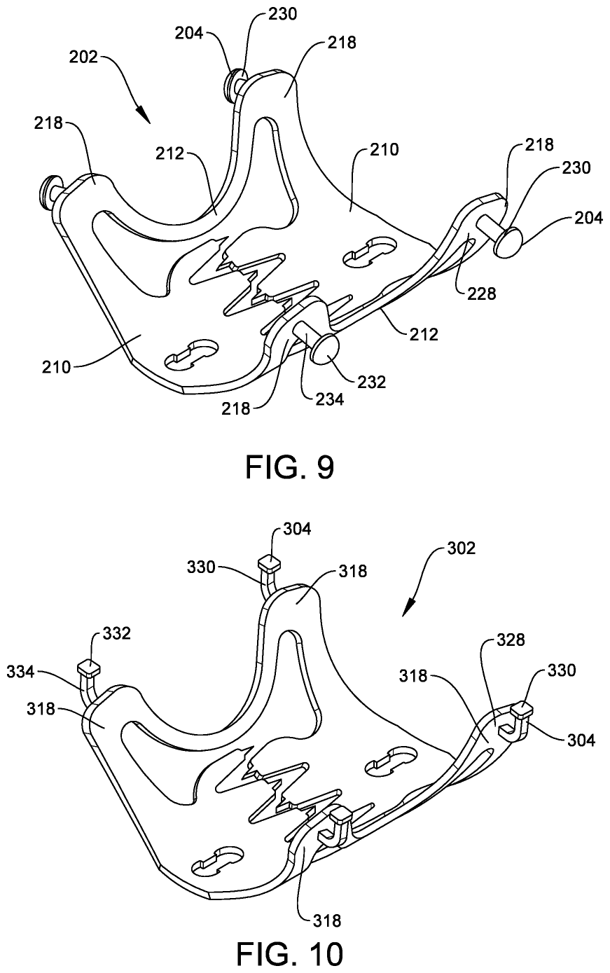

FIG. 9 shows a perspective view of a clip according to another exemplary embodiment of the present disclosure;

FIG. 10 shows a perspective view of a clip according to yet another exemplary embodiment of the present disclosure;

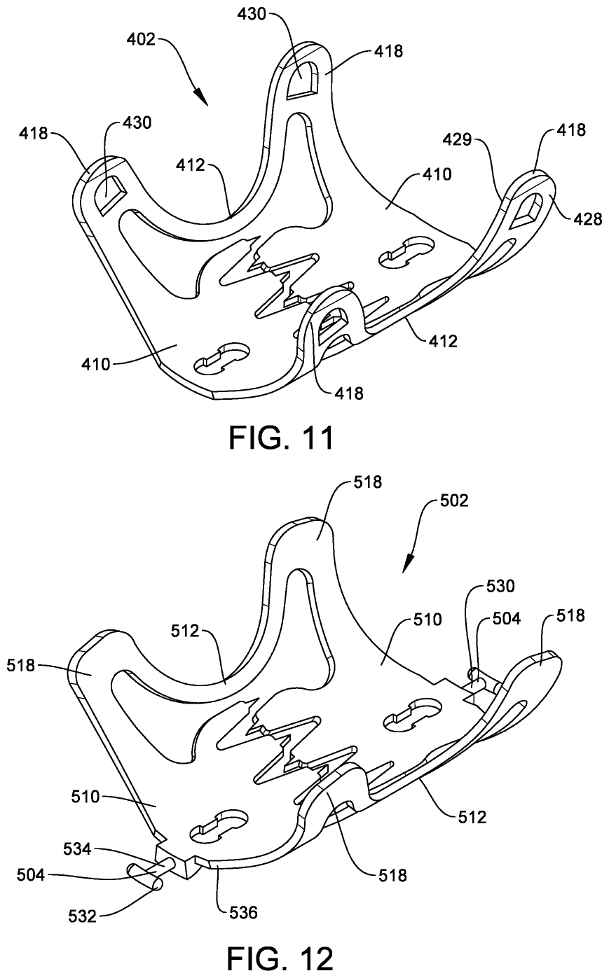

FIG. 11 shows a perspective view of a clip according to another exemplary embodiment of the present disclosure;

FIG. 12 shows a perspective view of a clip according to yet another exemplary embodiment of the present disclosure.

### DETAILED DESCRIPTION

The present disclosure may be further understood with reference to the following description and the appended drawings, wherein like elements are referred to with the same reference numerals. The present disclosure relates to an endoscopic clipping system and, in particular, relates to an over-the scope clipping system including a clip configured for removal after deployment/placement over tissue. Exemplary embodiments of the present disclosure describe an over-the-scope clip including features engageable via a through-the-scope device such as, for example, a snare or grasping forceps. The engaging features are configured to be engaged via the through-the-scope device to reopen the deployed clip and safely extract the clip from the body, without requiring prior treatment (e.g., severing of the clip) or adjustment of an endoscope from which the clip had been deployed.

As shown in FIGS. 1-8, a clipping system 100 comprises an over-the scope clip 102 including a plurality of engaging features 104 configured to facilitate extraction of the clip 102 after the clip has been deployed (clipped over tissue). In particular, the engaging features 104 are configured to be engaged via a through-the-scope device such as, for example, a snare device 106 so that applying tension to a loop 108 of the snare device 106 and, consequently, to the engaging feature(s) of the clip 102 moves the clip 102 reopens the clip 102 (i.e., moves the clip 102 from a closed configuration in which opposing jaws 110 of the clip 102 are in contact with and/or in close proximity to one another to grip tissue therebetween to an open configuration in which the jaws 110 are moved away from one another) to release the previously clipped tissue. The clip 102 may then be extracted from the patient's body without requiring any cutting of the clip 102 and/or any resection or dissection of tissue.

The clip 102, as shown in FIG. 1, includes a pair of jaws 110 that are connected to one another via hinges 112 so that the clip is movable between the open configuration, in which the jaws 110 are separated from one another, and the closed configuration in which the jaws 110 are moved toward one another to grip tissue therebetween. In an exemplary embodiment, each of the jaws 110 extends along a curve from a first end 114 to a second end 116. The curve of the jaws 110 is, in this embodiment, corresponds to a curvature of an outer surface of an end cap or other structure over which the clip 102 is to be mounted for deployment from an endoscope or other delivery device. The first ends 114 of the jaws 110 are connected to one another via a first one of the hinges 112 and the second ends 114 of the jaws 110 are connected to one another via a second one of the hinges 112.

In an exemplary embodiment, each of the hinges 112 is a living hinge and extends, when the jaws 110 are in the closed configuration, along a substantially C-shaped curve from the corresponding end of a first one of the jaws 110, toward a distal face of the jaws 110 to arc back toward the corresponding end of the other jaw 110. The hinges 112 are, in this embodiment, spring biased to bias the jaws 110 toward the closed configuration. It will be understood by those of skill in the art that the hinges 112 and/or jaws 110 of the clips 102 may be formed of any of a variety of materials so long as the hinges 112 bias the jaws 110 toward the closed configuration, as described above, and so that the bias is sufficiently strong to maintain the clip 102 in the closed configuration clipped over target tissue after the clip 102 has been finally deployed, as will be described in further detail below. In one example, portions of the clip 102 (e.g., the hinges 112) are formed of a shape memory alloy such as, for example, Nitinol to provide and/or add to the bias toward the closed configuration.

In an exemplary embodiment, ends 120 of each of the hinges 112 are connected to the first and second ends 114, 116 of a corresponding one of the jaws 110 via connecting portions 118. The hinges 112 are connected to the jaws 110 via connecting portions 118 so that there are four connecting portions 118 (118a, 118b, 118c, 118d), each of the connecting portions 118 at a corresponding one of the first and second ends 114, 116 of each of the jaws 110. In other words, as shown in FIG. 2, the first connecting portion 118a connects the first end 114 of a first one of the jaws 110a to a first one of the hinges 112a while the second connecting portion 118b connects the first end 114 of a second one the jaws 110b to the first one of the hinges 112a. The third connecting portion 118c connects the second end 116 of the first one of the jaws 110a to the second one of the hinges 112b while the fourth connecting portion 118d connects the second end 116 of the second one of the jaws 110b to the second one of the hinges 112b.

According to an exemplary embodiment, at least two of the connecting portions 118 include engaging features 104 configured to engage, for example, the loop 108 of the snare device 106 so that the loop 108 may apply tension to the engaging features 104 without slipping off the engaging features 104 (i.e., so that the tension may be applied to the engaging features to open the clip 102 as will be described below). Where only two connecting portions 118 include engaging features 104, the two connecting portions 118 must include a connecting portion 118 of each of the jaws 110. It will be understood by those of skill in the art, however, that in another embodiment, each of the connecting portions 118 (i.e., all four connecting portions 118) may include an engaging feature 104. In yet another exemplary embodiment, however, just one of the connecting portions 118 may include an engaging feature 104.

According to an exemplary embodiment, each engaging feature 104 includes a pair of notches (i.e., a first notch 122 and a second notch 124) along opposing sides of the connecting portion 118. A first one of the first notches 122 is formed along an exterior edge 123 of the connecting portion 118 on a side thereof that is in alignment with a surface of a first one of the jaws 110 that is opposite a tissue gripping surface of the first the jaw 110 and a second one of the second notches 124 is formed along the exterior edge 123 of the connecting portion 118 on a side of the connecting portion 118 facing the hinge 112. In an embodiment, the first and second notches 122, 124 are configured as a slot and are substantially aligned with one another so that a portion of the loop 108 drawn into the first notch 122 will, as it moves deeper into the first notch 122, be moving toward the second notch 124. The pair of the first and second notches 122, 124 are sized, shaped, and configured to receive and engage a portion of the loop 108 of the snare device 106 therein. Although the exemplary embodiments show and describe the connecting portions 118 as including pairs of the first and second notches 122, 124, in another embodiment, one or more of the connecting portions 118 may include a single notch (e.g., the first notch 122) in alignment with a corresponding one of the jaws 110.

Additionally, each of the jaws 110 may, in certain embodiments, include gripping features 126 such as, for example, teeth. The gripping features 126 may extend along each of the jaws 110, extending toward one another to grip tissue received between the jaws 110, when the clip 102 is in the closed configuration.

It will be understood by those of skill in the art, that although the clip 102 is biased toward the closed configuration, the clip 102 is maintained in the open configuration for insertion to a target area within the body. For example, the clip 102 may be stretched into the open configuration and positioned over an end cap which is, for example, mounted over a distal end of an endoscope so that the end cap holds the clip 102 in the open configuration until the clip 102 is deployed therefrom.

For example, in an exemplary embodiment, the clip 102 is opened and slip over the end cap so that each of the jaws 110 extends over opposing portions of the end cap with an exterior surface of the end cap holding the clip 102 in the open configuration. The clip 102, which is mounted over the end cap that is, in turn, mounted in a delivery device such as an endoscope, may be inserted to a target area adjacent to tissue to be clipped (e.g., by advancing the endoscope through a body lumen accessed via a natural body orifice). Target tissue is then drawn into the end cap (e.g., by applying suction to the endcap through a working channel of the endoscope) and the clip 102 so that the tissue within the endcap is received between the jaws 110. The clip 102 is then moved distally off of the end cap using any known deployment mechanism, so that, when the clip 102 moves distally off of the endcap, the bias of the hinges 112 draws the clip 102 to the closed configuration gripping the tissue that had been drawn into the end cap.

As will also be understood by those of skill in the art, the clip 102 may be moved distally off of the end cap toward the closed, clipping configuration via any of a number of control mechanisms. For example, in one embodiment, the clip 102 may be releasably coupled to one or more control elements that pull the clip 102 proximally and prevent the clip 102 from moving distally off of the cap. Distal ends of the control elements may be releasably coupled to the jaws 110 via, for example, the keyhole openings 111 extending therethrough. When a user of the system 100 (e.g., physician) determines that the desired portion of tissue has been drawn into the end cap, the user may release tension along these control elements permitting jaws of the clip to begin to close over a tapered surface of the end cap so that the bias of the hinges 112 and the closing of the jaws 110 pulls the clip 102 distally off of the endcap to clip the tissue received therebetween.

Once the clip 102 has been clipped over the tissue, the clip 102 may be finally deployed (as shown in FIG. 2) by releasing the clip 102 from the control elements as would be understood by those skilled in the art. Although the clip 102 has been described as being permitted to slide distally off of the end cap, in another embodiment, control elements releasably coupled to the clip 102 may be moved distally relative to the end cap to push the clip off the end cap (e.g., by applying a distally directed force to the control elements so that a column stiffness of the control elements applies a distally directed for the clip 102 pushing the clip 102 distally off of the endcap. It will also be understood by those of skill in the art that although the exemplary embodiments show and describe keyhole openings 111, the control elements may be releasably coupled to the clip 102 via any of a number of releasable connections so long as the control elements may be released therefrom upon clipping of the clip 102 over a tissue.

According to an exemplary method utilizing the system 100, upon determination that the clip 102 should be removed due to, for example, inaccurate placement, the snare device 106 may be inserted through a working channel of the endoscope. It will be understood by those of skill in the art that the snare device 106 may be inserted to the target area, without having to first remove the endoscope and/or adjust the endoscope in any way. According to one exemplary embodiment, once the snare device 106 has been inserted to the target area, the loop 108 of the snare device 106 is looped about the connecting portions 118 of the clip 102, as shown in FIG. 3, so that the loop 108 is received within the first notch 122 of each of the connecting portions 118. The loop 108 may then be tightened, as shown in FIG. 4, compressing each of the hinges 112 so that the connecting portions 118 of the jaws 110 are moved toward one another to move the clip 102 from the closed configuration to the open configuration.

As the jaws 110 move apart from one another toward the open configuration, the gripped tissue is released, allowing the user to draw the snare device 106, with the clip 102 still held via the loop 108, away from the tissue, as shown in FIG. 5. After the clip 102 has been moved away from the released tissue, tension on the loop 108 may be loosened slightly to allow the clip 102 to revert to the closed configuration. The clip 102 may then be drawn proximally against the distal end of the endoscope so that the endoscope may be withdrawn from the body to remove the clip 102 from the body.

According to another exemplary method of use for the system 100, the user may remove the deployed clip by looping the loop 108 of the snare device 106 over two of the connecting portions. In one embodiment, as shown in FIG. 6, the loop 108 is positioned over two diagonally positioned connecting portions 118 (118a, 118c). For example, the loop 108 may be positioned so that the loop 108 extends over a first connecting portion 118a at the first end 114 of the first one of the jaws 110 and a third connecting portion 118c at the second end 116 of the second one of the jaws 110. The loop 108 may be received in the pair of the first and second notches 122, 124 of each of the first and second connecting portions 118a, 118b.

The loop 108 is then tightened to draw the two connecting portions 118 toward one another, forcing the jaws 110 away from one another, from the closed configuration to the open configuration. Thus, the gripped tissue is released permitting the clip 102 to be removed from the body with the endoscope, as described above. It will be understood by those of skill in the art that since the clip 102 is not required to be cut, the clip 102 and, thus, does not include sharp edges, the clip 102 may be removed from the body, along with the endoscope, without causing damage to any surrounding tissue.

In another exemplary method, as shown in FIG. 7, the loop 108 of the snare device 106 is positioned over opposing connecting portions (118a, 118b). For example, the loop 108 may be positioned so that the loop 108 extends over the first connecting portion 118a at the first end 114 of the first one of the jaws 110 and the second connecting portion 118b at the first end 114 of the second one of the jaws 110. The loop 108 is received within the first notch 122 of each of the first and second connecting portions 118a, 118b so that when the loop 108 is tightened, the first and second connecting portions 118a, 118b are drawn toward one another, compressing the hinge 112 extending therebetween so that the jaws 110 are separated from one another to release the gripped tissue. As described above, the clip 102 may then be removed from the body with the endoscope.

According to yet another exemplary method, as shown in FIG. 8, the loop 108 of the snare device 106 may be looped over a single one of the connecting portions 118 so that the loop 108 is received within the first and second notches 122, 124. The loop 108 may be tightened so that the snare device 106 is able to grasp the clip 102 thereby. Thus, drawing the snare device 106 proximally will correspondingly draw the clip 102 proximally relative to the tissue, pulling the clip 102 off of the tissue. It will be understood by those of skill in the art that this method may be particularly useful when the clip 102 has been superficially clipped over the tissue. Once the clip 102 has been pulled away from the tissue (e.g., tearing a portion of the tissue), the clip 102 may be removed from the body, as described above.

Although the engaging features 104 of the clip 102 are described and shown as including the first and second notches 122, 124 along connecting portions 118 thereof, it will be understood by those of skill in the art that the engaging features 104 may include any of a number of different structural features so long as the engaging features 104 are configured to be grasped, gripped or otherwise engaged via a through-the-scope-device such as, for example, a snare device or grasping forceps, which may facilitate removal of the clip 102 thereby.

In an exemplary embodiment, as shown in FIG. 9, for example, a clip 202 according may be substantially similar to the clip 102, described above, comprising a pair of opposing jaws 210 connected to one another via hinges 212 so that the jaws 210 are movable relative to one another between an open configuration, in which the jaw 210 are separated from one another, and a closed configuration, in which the jaws 210 are moved toward one another to grip tissue. Similarly to the clip 102, the hinges 212 are connected to the jaws 210 via connecting portions 218 including engaging features 204 which may be engaged via a through-the-scope device such as, for example, a snare device or grasper forceps, for extraction of the clip 202 from the body. Rather than notches, however, the engaging features 204 of this embodiment include a protrusion 230 extending from an exterior surface 228 of the clip 202 (i.e., a surface of the clip 202 which, when the clip 202 is mounted over an end cap of an endoscope, faces away from the end cap).

The protrusion 230 is, in this embodiment, configured as a pin including a stem 234 extending from the exterior surface to an enlarged head 232 at the free end thereof. The enlarged head 232 may take any of a variety of shapes and configurations so long as the enlarged head 232 facilitates a looping, grasping, or other engagement using a through-the scope device such as a snare device or grasper forceps so that sufficient tension may be applied thereto to open the clip 202. In an exemplary embodiment, the stem 234 extends substantially perpendicularly relative to the exterior surface 228.

According to an exemplary method of use for the clip 202, a loop of a snare device as described above is manipulated to engage the stems 234 of one or more of the protrusions 230, using any of the exemplary methods described above with respect to the system 100. For example, where the snare device is used to loop two or more of the protrusions 230, tightening the loop will compress at least one of the hinges 212 to move the clip 202 from the closed configuration to the open configuration to release gripped tissue. It will be understood by those of skill in the art that the enlarged head 232 facilitates engagement of the stem 234 with the loop by preventing the loop from sliding off of the stem 234 when tension is applied to the loop. In another example where the snare device is used to loop one of the protrusions 230, the loop of the snare device is tightened around the looped protrusions so that tension applied to the snare device is applied to the clip 202 to open pull the clip 202 off of the clipped tissue.

According to another exemplary method of use for the clip 202, grasper forceps may be used to grasp one or more protrusions 230 to extract a deployed clip 202, in a manner substantially similar to the methods described above in regard to the use of snare devices. In one embodiment, the grasper forceps may be used to grasp two protrusions of the jaws 210 so (i.e., with one jaw of the forceps grasping a protrusion at an end of a hinge coupled to a first one of the jaws 210 while the other jaw of the forceps engages a protrusion at the other end of the same hinge (i.e., at the end of the same hinge that is attached to the other jaw 210). Drawing the jaws of the forceps toward one another then pulls these protrusions toward one another to move the clip 202 from the closed configuration to the open configuration.

As discussed above with respect to the system 100, the forceps may be used to grasps two protrusions 230 directly opposing one another (e.g., at first ends 214 of the jaws 210) or diagonal to one another (e.g., the single protrusion 230 at the first end 214 of one of the jaws 210 and one protrusion at a second end of the second one of the jaws 210). In another embodiment, grasper forceps may be used to grasp the single protrusion 230 to pull the clip 202 off the clipped tissue.

As shown in FIG. 10, a clip 302 according to another exemplary embodiment is substantially similar to the clip 202, comprising jaws 310 connected to one another via connecting portions 318 including engaging features 304, each of which may be configured as a protrusion 330 extending from an exterior surface 328 of the clip 302. Similarly to the protrusion 230, the protrusion 330 includes an enlarged head 332 connected to the exterior surface 328 via a stem 334. The stem 334, however, is bent and/or curved to extend in a substantially L-Shaped configuration, so that the protrusion 330 extends in a proximal direction—e.g., a direction away from tissue gripped by the clip 302.

The clip 302, once deployed/placed, may be extracted from a clipped tissue in a manner substantially similar to the clip 202, using any of the methods described above with respect to the system 100 and the clip 202. In particular, any of a variety of through-the-scope devices such as, for example, a snare device or grasper forceps may be utilized to grasp and/or engage one or more of the protrusions 330 to open, release from tissue and extract the clip 302, as described above.

According to another exemplary embodiment, as shown in FIG. 11, a clip 402 according to another exemplary embodiment, is substantially similar to the clips described above, comprising jaws 410 movable between an open configuration, in which jaws 410 thereof are separated from one another, and a biased closed configuration, in which the jaws 410 are moved toward one another, via hinges 412 connected to the jaws 410 at connecting portions 418. In this embodiment, however, rather than protrusions or notches, each of the connecting portions 418 includes an opening 430 extending therethrough, from an interior surface 429 of the clip 402 (i.e., a surface of the clip 402 which, when the clip 402 is mounted over an end cap of the endoscope, faces toward the end cap) to the exterior surface 428 (i.e., a surface of the clip 402 which, when the clip 402 is mounted over the end cap, faces away from the end cap). Those skilled in the art will understand that the openings 430 may take any of a variety of configurations so long as the openings 430 are configured to facilitate a grasping of the connecting portions 418 via, for example, grasper forceps, to move the clip 402 from the closed configuration toward the open configuration in the manner described above.

Upon deployment/placement, the clip 402 may be extracted from the body using, for example, grasper forceps, as described above. In particular, the grasper forceps may be used to grasp one or more of the openings 430 to either pull the clip 402 from the clipped tissue or draw opposing connecting portions 418 (e.g., diagonally positioned or directly opposing) toward one another to move the clip 402 from the closed configuration to the open configuration, as described above.

According to another exemplary embodiment, as shown in FIG. 12, a clip 502 is substantially similar to the clips described above, comprising jaws 510 connected to one another via hinges 512 so that the jaws 510 are movable relative to one another between an open configuration, in which the jaws 510 are separated from one another, and a closed configuration, in which the jaws 510 are moved toward one another. The clip 502 also includes engaging features 504 via which a deployed/placed clip 502 may be extracted from a body using, for example, a snare device and/or grasper forceps. Each of the engaging features 504 of the clip 5023, rather than being positioned along connecting portions 518 which connect the hinges 512 to the jaws 510, extend from an end of a corresponding one of the jaws 510 opposite the tissue gripping end of each of the jaws 510 (i.e., opposite the surfaces of the jaws 510 between which tissue is gripped).

In an exemplary embodiment, each of the engaging features 504 includes a protrusion 530 extending from, for example, an edge 536 of the jaw 510 that faces away from the other one of the jaws 510. In particular, each protrusion 530 extends from the edge 536 of a corresponding one of the jaws 510 in a direction extending away from the other jaw 510, so that the protrusion 530 extends away from gripped tissue so that it will be exposed to facilitate gripping via the grasper forceps or other device. Similarly to the protrusions 230, 330 described above with respect to the clips 202, 302, respectively, the protrusion 530 include an enlarged head 532 (or other engaging structure) connected to the edge 536 via a stem 534. As described above with respect to the clips 202 and 302, the protrusion 530 facilitate the extraction of the clip 502 utilizing a through-the-scope device such as a snare device or grasper forceps, using any of the extraction methods described above.

It will be apparent to those skilled in the art that various modifications may be made in the present disclosure, without departing from the scope of the disclosure. Furthermore, those skilled in the art will understand that the features of any of the various embodiments may be combined in any manner that is not inconsistent with the description and/or the functionality of the embodiments.

1-15. (canceled)

16. An over-the-scope clip, comprising:
a first jaw and a second jaw, each of the first and second jaws extending along a curve from a first end to a second end;
a first hinge connecting the first ends of the first and second jaws and a second hinge connecting the second ends of the first and second jaws so that the first and second jaws are movable between at open configuration, in which the first and second jaws are separated from one another, and a closed configuration, in which the first and second jaws are drawn toward one another to grip tissue therebetween, at least one of the first and second hinges being biased to draw the clip toward the closed configuration; and
a connecting portion connecting an end of each of the first and second hinges to a corresponding one of the first and second ends of each of the first and second jaws, the connecting portion including an engaging feature configured to be engaged via a through-the scope device to extract the clip from a tissue over which it has been clipped upon a deployment thereof.

17. The clip of claim 16, wherein the engaging feature includes a first notch extending along a portion of an exterior edge of the connecting portion in alignment with a corresponding one of the first and second jaws.

18. The clip of claim 17, wherein the engaging feature includes a second notch extending along a portion of the exterior edge of the connecting portion in alignment with a corresponding one of the first and second hinges.

19. The clip of claim 18, wherein the first and second notches are configured as slots which are aligned with one another.

20. The clip of claim 16, wherein the engaging feature is a protrusion extending from an exterior surface of the connecting portion, the protrusion including an enlarged head connected to the exterior surface via a stem.

21. The clip of claim 20, wherein the stem extends substantially perpendicular to the exterior surface.

22. The clip of claim 20, wherein the stem includes an L-shaped bend.

23. The clip of claim 16, wherein the engaging feature includes an opening extending through the corresponding one of the connecting portions.

24. The clip of claim 16, wherein each of the first and second hinges includes a curve extending toward and between the first and second jaws.

25. The clip of claim 16, wherein each of the first and second jaws include a plurality of teeth configured to grip tissue received therebetween.

26. An over-the-scope clip, comprising:
a first jaw and a second jaw, a first end of the first jaw being connected to a first end of the second jaw via a first hinge and a second end of the first jaw being connected to a second end of the second jaw via a second hinge so that the first and second jaws are movable between an open configuration, in which the first and second jaws are separated from one another, and a closed configuration, in which the first and second jaws are drawn toward one another to grip tissue therebetween, at least one of the first and second hinges being biased to draw the clip toward the closed configuration; and
a first engaging feature extending from an edge of the first jaw which faces away from the second jaw and a second engaging feature extending from an edge of the second jaw which faces away from the first jaw.

27. The clip of claim 26, wherein each of the first and second engaging features include a protrusion including an enlarged head connecting to the edge of a corresponding one of the first and second jaws via a stem.

28. The clip of claim 26, wherein each of the first and second jaws extend along a curve corresponding to a surface of an end cap of an endoscope over which the clip is to be mounted.

29. The clip of claim 26, wherein each of the first and second hinges includes a curve extending toward and between the first and second jaws.

30. The clip of claim 26, wherein each of the first and second jaws include a plurality of teeth configured to grip tissue received therebetween.

31. A method for extracting a deployed over-the-scope clip from a body, comprising:
inserting a snare device through a working channel of an endoscope via which the clip was clipped over a tissue and deployed;
looping a wire of the snare device over two or more engaging features of first and second jaws of the clip so that the wire engages the one or more engaging features, the clip including a first hinge connecting first ends of the first and second jaws and a second hinge connecting second ends of the first and second jaws, ends of the first and second hinges connected to a corresponding one of the first and second ends of the first and second jaws via connecting portions including the engaging features;
tightening the wire to draw the two or more engaging features toward one another so that one of the first and second hinges is compressed to move the clip from a closed configuration, in which the first and second jaws are moved toward one another to clip a tissue therebetween, toward an open configuration, in which the first and second jaws are moved away from one another to release the tissue gripped therebetween;
drawing the clip proximally away from the released tissue and against a distal end of the endoscope; and
removing the clip from the body, simultaneously with the endoscope.

32. The method of claim 31, wherein each of the engaging features includes a first notch extending along a portion of an exterior edge of the connecting portion in alignment with a corresponding one of the first and second jaws and a second notch extending along a portion of the exterior edge of the connecting portion in alignment with a corresponding one of the first and second hinges.

33. The method of claim 31, wherein the wire is looped over four connecting portions.

34. The method of claim 31, wherein the wire is looped over the engaging features of two diagonally positioned connecting portions, a first one of the connecting portions at the first end of the first jaw and a second one of the connecting portions at the second end of the second jaw.

35. The method of claim 31, wherein the wire is looped over the engaging features of two opposing connecting portions, a first one of the opposing connecting portions at the first end of the first jaw and a second one of the opposing connecting portions at the first end of the second jaw.
